(19) United States
(12) Patent Application Publication
(10) Pub. No.: US 2023/0364424 A1
(43) Pub. Date: Nov. 16, 2023

(54) AUGMENTING REALITY

(71) Applicant: Cochlear Limited, Macquarie University, NSW (AU)

(72) Inventors: Nicholas FEENEY, Kullavik (SE); Marcus ANDERSSON, Goteborg (SE); Kenneth OPLINGER, Macquarie University, NSW (AU)

(21) Appl. No.: 18/041,029

(22) PCT Filed: Aug. 13, 2021
(86) PCT No.: PCT/IB2021/057476

§ 371 Date: Feb. 8, 2023

Related U.S. Application Data
(60) Provisional application No. 63/065,790, filed on Aug. 14, 2020.

(51) Int. Cl. A61N 1/36; G16H 40/67; G16H 20/30

(52) U.S. Cl. CPC A61N 1/36139; A61N 1/36038; G16H 40/67; G16H 20/30; H04R 2225/41; H04R 25/606

(57) ABSTRACT

Disclosed examples include monitoring consumption behavior of a recipient of a sensory prosthesis (e.g., a cochlear implant). The sensory prosthesis identifies specific sounds (e.g., the sounds of vomiting, snoring, opening a beer bottle, lighting a cigarette, or taking medication from a blister, among others) or visuals and record data regarding the frequency, timing, intensity, or other characteristics of the behavior. The recorded data can then be analyzed by the recipient or caregivers. The sensory prosthesis further adjusts its sensory output to enhance, degrade, or otherwise modify the recipient's perception of the consumption behavior.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an example, there is a method including: detecting a consumption behavior of a recipient of a sensory prosthesis; and adjusting stimulation provided by the sensory prosthesis to adjust the consumption behavior.

In another example, there is a system including a sensory prosthesis of a recipient; a microphone; a movement sensor; and a computing device. The computing device is configured to: receive, from the microphone and the movement sensor, consumption behavior indicia regarding the recipient; determine a consumption behavior of the recipient based on the consumption behavior indicia; and act on the determined behavior to adjust the consumption behavior.

In a further example, there is a computer-readable medium having instructions stored thereon that, when executed by one or more processors cause the one or more processors to: log a consumption behavior by a recipient of a sensory prosthesis based on data obtained by the sensory prosthesis; and display information regarding the consumption behavior.

### BRIEF DESCRIPTION OF THE DRAWINGS

The same number represents the same element or same type of element in all drawings.

FIG. 1 illustrates a system with which one or more techniques described herein can be implemented.

FIG. 2 illustrates example behavior indicia.

FIG. 3 illustrates an example method.

FIG. 4 illustrates an example computing device configured to perform a method that includes operations.

FIG. 5 illustrates example memory having instructions stored thereon that, when executed by one or more processors, cause the one or more processors to perform a method including one or more operations.

FIG. 6 illustrates an example artificial intelligence framework usable with examples herein.

FIG. 7 illustrates an example of a suitable computing system with which one or more of the disclosed examples can be implemented.

FIG. 8 is a functional block diagram of an implantable stimulator system that can benefit from technologies described herein.

FIG. 9 illustrates an example cochlear implant system that can benefit from technologies disclosed herein.

FIG. 10 is a view of an example of a percutaneous bone conduction device that can benefit from use of the technologies disclosed herein.

FIG. 11 illustrates a retinal prosthesis system that comprises an external device, a retinal prosthesis, and a mobile computing device.

### DETAILED DESCRIPTION

Disclosed examples include the use of sensory prostheses (e.g., auditory prostheses and visual prostheses) to monitor consumption behavior by a recipient of the sensory prosthesis. For instance, the sensory prosthesis identifies specific sounds (e.g., the sounds of opening a beer bottle, lighting a cigarette, vomiting, snoring, or taking medication from a blister, among others) or visuals and records data regarding the frequency, timing, intensity, or other characteristics of the identified sounds or visuals. The recorded data is then analyzed by the recipient or caregivers.

In addition to or instead of logging the behavior, the sensory prosthesis is used to alter a recipient's experience of consumption behaviors, such as eating or drinking. Further, examples include tracking consumption behaviors, encouraging healthy consumption behaviors, and discouraging unhealthy consumption behaviors.

An example sensory prosthesis detects when a recipient is engaging in a consumption behavior and, in response, causes sensory output (e.g., audio or visual output) configured to make the recipient consume faster, consume slower, or change how the recipient perceives what they are consuming. As a specific example, an auditory prosthesis detects that a recipient is eating a celery stalk, and the auditory prosthesis plays a sound that enhances the crunchy sound of the celery stalk to make the recipient experience the crunchiness of the stalk in a way that enhances the pleasurability of eating the healthy celery stalk. In a further example, the auditory prosthesis detects that the recipient is eating potato chips, and the auditory prosthesis plays a sound that inhibits the crunchy sound of the potato chip to make the recipient experience the potato chip in a way that decreases the pleasurability of eating the unhealthy potato chips. In another example, a visual prosthesis detects that a recipient is looking at a buffet of healthy and unhealthy food, and in response the visual prosthesis increases the saturation of the healthy food and decreases the saturation of the unhealthy to affect how the recipient perceives the food to make the healthy food look more appetizing than the unhealthy food. Thus, disclosed techniques can be used to change how the recipient experiences consumption behavior (e.g., eating a chip or celery stalk) or objects associated with consumption behavior (e.g., food items themselves) to encourage healthy behavior and discourage unhealthy behavior. Consumption behavior need not be limited to food. Consumption behavior includes drinking (e.g., encouraging consumption of water and discouraging consumption of alcohol or soda) and smoking/vaping (e.g., discouraging such activities), among others. The recipient's perception of non-consumption behavior such as vomiting (e.g., as a result of bulimia), breathing, or exercise can also be affected using techniques described herein.

The sensory prosthesis can be configured to not only enhance or detract from an existing quality of the behavior (e.g., enhancing or muting a sound that would normally be produced by eating a chip), but in addition or instead add a sensory percept that is not naturally tied to the consumption behavior. For instance, responsive to detecting that the recipient is smoking a cigarette, an auditory prosthesis produces a sound that makes the recipient feel discomfort to associate smoking with discomfort or stimulates the recipient's vestibular system in a way that makes the recipient feel dizzy.

While in many examples, the sensory prosthesis alters the experience of the consumption behavior in a way consistent with a primary modality of a sensory prosthesis (e.g., an auditory prosthesis can affect the aural perception of the behavior and a visual prosthesis can affect the visual perception of the behavior), the sensory prosthesis can affect the perception in other ways. In an example, a visual prosthesis has a speaker that produces audio to alter a perception of the consumption behavior. In another example, an auditory prosthesis has a haptic or vibratory driver that provides vibrations to a user that affects the perception of the consumption behavior. In further examples, a sensory prosthesis causes another device to alter the recipient's perception.

Example devices can use any of a variety of different kinds of inputs. Implanted or external microphones of the sensory prosthesis are used to detect: ambient sound (e.g., sounds of restaurants or speech), external sound cues (e.g., the sound of crunching, the sound of lighting a cigarette, the sound of a beer bottle or can being opened, the sound of a toilet, or the sound of vomiting), or internal sound cues (e.g., stomach sounds, hunger sounds, jaw tensing sounds, chewing sounds, swallowing sounds, or breathing sounds). A sensory prosthesis uses an accelerometer to detect head movement (e.g. indicating alcohol intoxication, vomiting, or eating movements), hand movement (e.g. fork toward the recipient's mouth), or general movement (e.g. walking, riding, driving, or falling). A sensory prosthesis uses a location sensor to detect a location of a recipient (e.g., that the recipient is in a restaurant). A sensory prosthesis obtains a manual setting or input (e.g. pre-set times, tactile on/off, speech controlled on/off, dieting program), such as one that indicates that the recipient is engaging in a particular activity and would like their sense of the activity to be altered (e.g., the recipient manually engaging an enhance-consumption mode). A scene classifier of the sensory prosthesis is used to detect a current environment in which the sensory prosthesis is operating (e.g., to detect a sound or visual environment). The sensory prosthesis obtains data from sensors in other devices (e.g. to detect glucose level with a separate blood glucose monitor, blood-alcohol level detected by a separate blood-alcohol level monitor, blood-sugar level, blood-oxygen level). The sensory prosthesis obtains data from software or services of other devices (e.g. a calendar service to detect events, data from a smart scales application to detect weight, data from a calorie counter application to detect calorie intake/goals, bank account data from a banking application to determine how much money the recipient spent at a given time/location or on certain items, such as food or drink). A proximity sensor (e.g., using near-field communication or another technology) detects the presence, absence, or proximity of another device (e.g. to detect a wrist accessory's proximity to hearing device, to detect proximity of a utensil to a mouth, or to detect proximity with respect to a medication jar). Other techniques are also usable in addition to or instead of those described above.

The obtained data is processed using any of a variety of techniques. Example processing includes interpreting the input, determining the type of input (e.g., sound) that is present, understanding what the input is, determining what the input means, storing the input, analyzing the input, determining actions to be taken based on the input, determining how to tweak the input (e.g., to cause the input to cause a particular kind of output, other techniques, or combinations thereof). In examples, the processing includes performing big data analysis of input or norms, determining best action for outcomes. Machine learning or artificial intelligence can be used (e.g. to learn responses based on effectiveness of responses to input or learn habits, such as based on responses to questions to the user “are you eating?”). Examples of processing the input include performing social sharing, sharing data with one or more databases, or performing data logging (duration, frequency, etc.). In some examples, the processing has a feedback loop with the input and the output such that effective deterrents to particular behaviors can be found.

In some examples, the output includes a request for confirmation from the recipient (or another) based on a prediction based on the processing. For instance, the sensory prosthesis determines that, proximate the time a recipient is due to take medication, a swallowing sound is detected. As a result of detecting the swallowing consumption behavior, the sensory prosthesis predicts that the recipient took medication. The sensory prosthesis then asks the recipient to confirm that the recipient did indeed take the medication. A response from the recipient is obtained (e.g., confirming or denying that the recipient took the medication) and logged. In certain implementations, the result is used to train an artificial intelligence framework (e.g., using the data obtained regarding the purported consumption behavior as the data and using the response from the recipient as a label for the data usable in training).

Based on the processing, the sensory prosthesis takes any of a variety of actions. For example, data and analysis (e.g. visual representation of data or analysis of usage time) is presented to the recipient or others. Data is entered into a recording problem (e.g., to put the data into long-term storage, such as a database configured for such a purpose). As a result of the processing, the output of the prosthesis is adjusted. The adjustment takes any of a variety of forms depending on how the prosthesis is configured and the desired outcome. For example, the adjustment isolates the sound of the recipient performing the consumption behavior (e.g., for further processing), adjusting the isolated sound, and stimulate the recipient with the adjusted sound. Example adjustments include an increased level for high frequency, middle frequency, or low frequency components of the sound. Example adjustments are used to make the sound a beverage can being opened less appealing or to suppress fizzing sound of carbonated drinks. Where the sound is internal body noise (e.g., as detected via a subcutaneous microphone), an example modification is to include less level reduction for the high frequency components than the low frequency components. This can be in contrast to typical internal body noise compensation schemes, which are typically configured to reduce body noise across all frequencies.

An example output is used to isolate the sound of the recipient eating or stomach rumbling and remove or reduce such sounds from the output of the prosthesis. Some individuals (e.g., having an eating disorder) may benefit from hearing their stomach rumbling and as such an auditory prosthesis can enhance (or at least not suppress) sounds of a stomach rumbling. In addition to or instead of changing the perception of the consumption behavior itself, an example sensory prosthesis changes a sensory-scape of the recipient based on the consumption behavior to compliment or detract from the recipient's consumption behavior (e.g. by providing a soothing, disturbing, or disrupting environment). In examples, the output is provided by a device other than the sensory prosthesis. For instance, another device provides text prompts or notifications based on a consumption behavior or lack thereof (e.g., notifications such as “this is your third cigarette today” or “you need to eat lunch”). Other example prompts or notifications include beeps, music, speech, flashes, vibrations, or light. Text prompts and notifications need not be provided solely to the recipient but additionally or instead be provided to a caregiver or medical professional (e.g. “your child hasn't eaten lunch yet” or “your patient vomited 1 hour after eating today”). In examples, an action enters data regarding the consumption behavior into a data recording program (e.g., by recording movements, sounds and other inputs to precisely identify duration of meals, number of drinks, eating speed and so on until the system senses strong indicators that the meal or other event is complete). Data for a completed event is then stored and analyzed in connection with other events.

An example method includes obtaining behavior input from any of a variety of input devices (e.g., as described above). On a periodic basis, relevant sensors are scanned for the indication. To improve accuracy of behavior detection, in certain examples processing includes confirmation of the behavior via one or more other sources. For instance, a location sensor (e.g., a satellite-based location system) indicates that the recipient is at a restaurant, and the microphone and accelerometer then confirm that the recipient is performing actions consistent with eating (e.g., rather than visiting the restaurant for some other reason). A hearing prosthesis further detects sound consistent with eating (e.g., internal body noise consistent with chewing and swallowing food). These indicia of eating are used to determine that the recipient is eating, and optional confirmation is obtained from the recipient (e.g., via a notification on a mobile device of the recipient). The processing is performed by the sensory prosthesis itself, by a mobile device (e.g., phone), by a server, by other devices or by combinations thereof. Responsive to the processing being completed (e.g., determining that the recipient is eating), an action is performed based on the result of the processing. The actions that are taken include one or more of the actions described above.

Further examples include a sensory prosthesis configured to detect consumption behaviors, such as eating, drinking, or smoking, with a subcutaneous microphone. In addition or instead, the sensory prosthesis detects consumption behaviors with a microphone (e.g., implanted or external) and a motion detection module (e.g., embedded in the prosthesis or other body worn device). In addition or instead, the sensory output of the sensory prosthesis is adjusted in response to detecting consumption behaviors to adjust such behaviors. In addition or instead, the sensory output of a sensory prosthesis is adjusted in response to a specific sensory input, such as a certain sound, to encourage or discourage consumption behaviors. In addition or instead, the sensory prosthesis logs and displays (e.g., directly or via another device) consumption behaviors with a sensory prosthesis, which is configured to detect consumption behavior details, and a phone, which is configured to display some or all of the details.

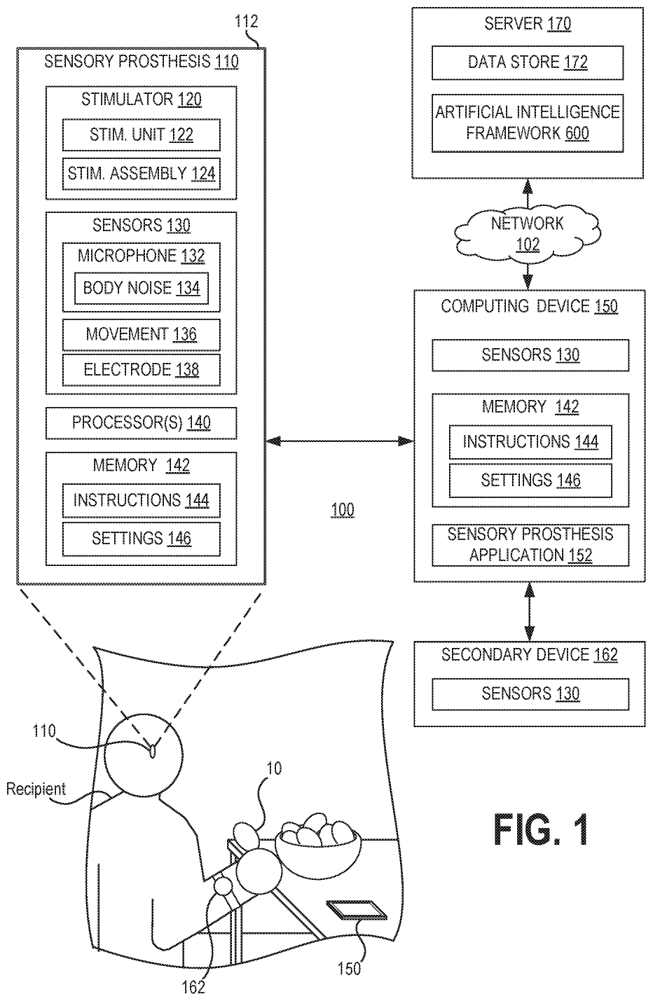

An example system is described in FIG. 1.

### Example System

FIG. 1 illustrates an example system 100 with which one or more techniques described herein are implemented. As illustrated, the system 100 includes a sensory prosthesis 110 of a recipient (e.g., implanted in or worn by the recipient), a computing device 150, and a secondary device 162. The computing device 150 is connected to a server 170 over a network 102. The network 102 is a computer network, such as the Internet, which facilitates the electronic communication of data among computing devices connected to the network 102.

FIG. 1 further illustrates a behavior 10 in which the recipient is engaging. In the illustrated example, the behavior 10 is eating potato chips from a bowl. The term “behavior” encompasses how the recipient of the sensory prosthesis 110 behaves and includes not only a long-term manner in which the recipient behaves (e.g., habits) but also individual actions of the recipient irrespective of whether such actions are or are not manifestations of habits of the person. In addition to behaviors in general, examples herein can relate to consumption behavior 10. The term “consumption” as used herein typically encompasses the recipient taking in something. Typically, consumption encompasses the recipient eating food or drinking a beverage. Consumption further refer to the recipient smoking or vaping a substance. Examples of consumption behaviors include an eating behavior, a drinking behavior, a vaping behavior, and a smoking behavior, among others.

### System—Sensory Prosthesis

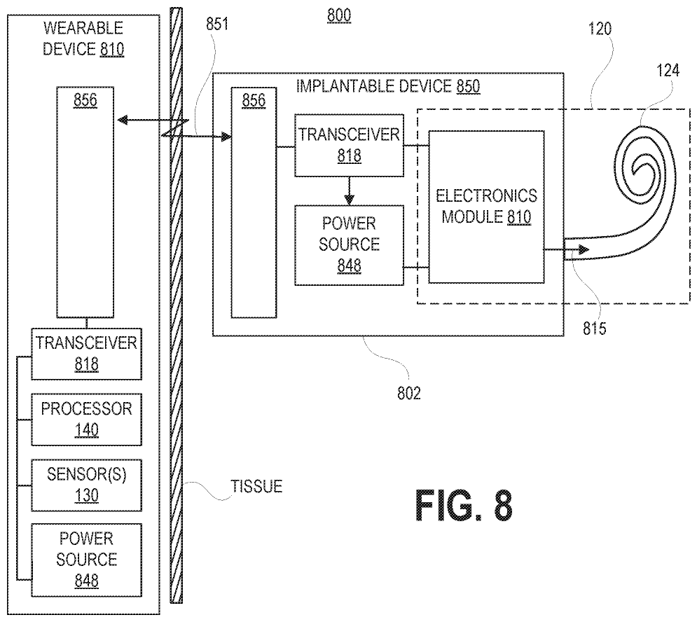

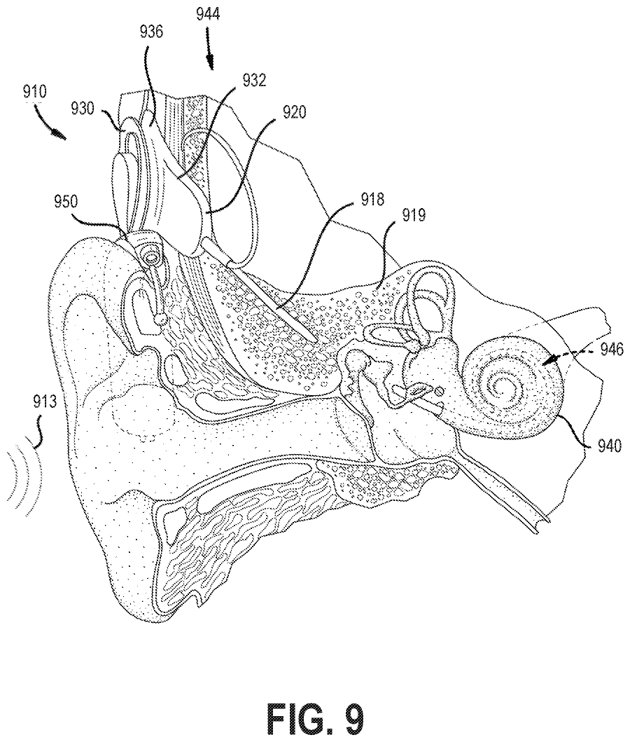

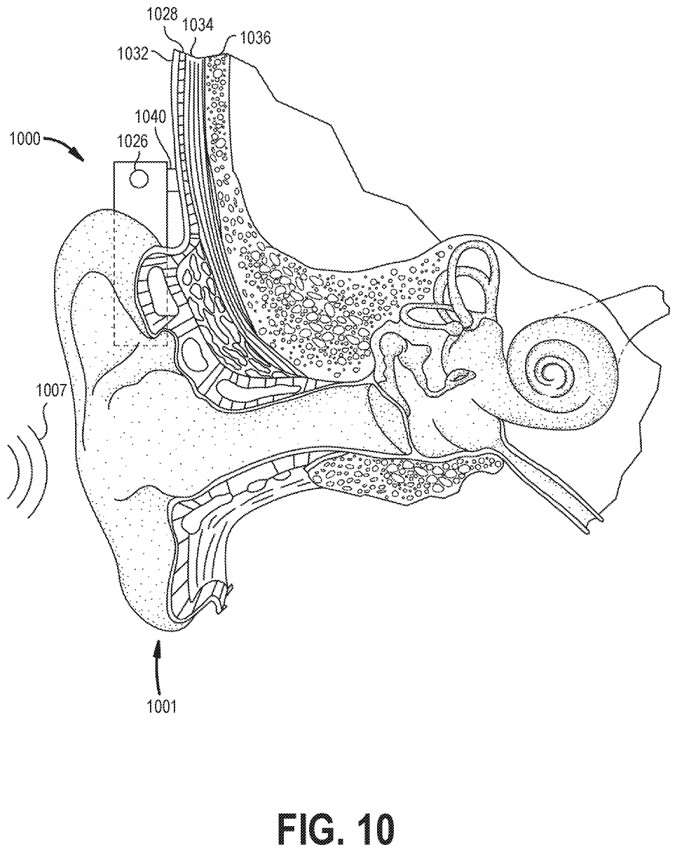

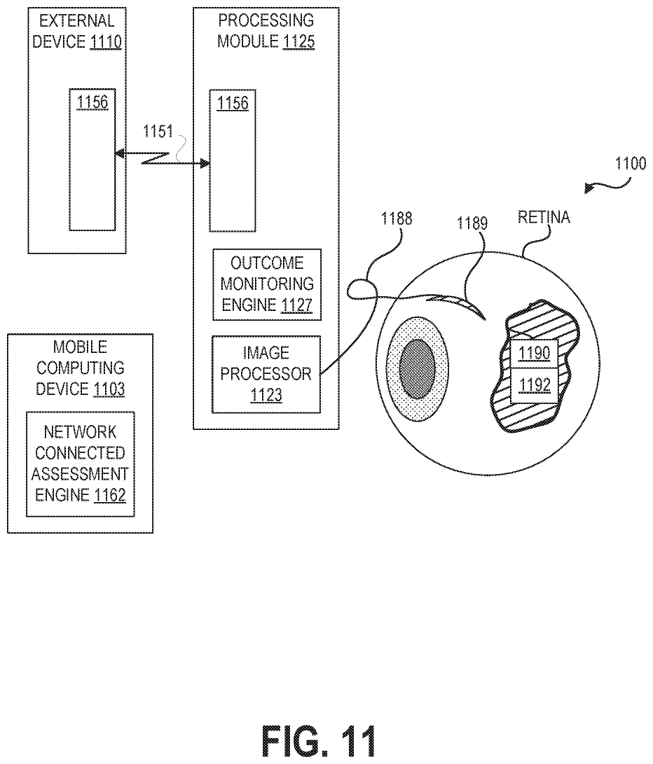

The sensory prosthesis 110 is an apparatus relating to one or more of the recipient's senses. For example, the sensory prosthesis 110 is a prosthesis relating to one or more of the five traditional senses (vision, hearing, touch, taste, and smell) and/or one or more additional senses. For ease of understanding, in many examples disclosed herein, the sensory prosthesis 110 is an auditory prosthesis configured to treat a hearing-impairment of the recipient, but many examples specific to auditory prostheses can be applicable to other kinds of sensory prostheses. Where the sensory prosthesis 110 is an auditory prosthesis, the sensory prosthesis 110 can take a variety of forms including a cochlear implant, an electroacoustic device, a percutaneous bone conduction device, a passive transcutaneous bone conduction device, an active transcutaneous bone conduction device, a middle ear device, a totally-implantable auditory device, a mostly-implantable auditory device, an auditory brainstem implant device, a hearing aid, a tooth-anchored hearing device, other auditory prostheses, and combinations of the foregoing (e.g., binaural systems that include a prosthesis for a first ear of a recipient and a prosthesis of a same or different type for the second ear). In examples, the sensory prosthesis 110 is or includes features relating to vestibular implants and visual prostheses (e.g., bionic eyes). Example implementations of the sensory prosthesis 110 are described in more detail in relation to FIG. 8 (showing an implantable stimulation system), FIG. 9 (showing a cochlear implant), FIG. 10 (showing a bone conduction device), and FIG. 11 (showing a retinal prosthesis). Technology disclosed is implementable with other devices and systems used in for example, such as sleep apnea management devices, tinnitus management devices, and seizure therapy devices. Technology disclosed herein can be used with consumer auditory devices (e.g., a hearing aid or a personal sound amplification product).

The illustrated example sensory prosthesis 110 includes a housing 112, a stimulator 120, one or more sensors 130, one or more processors 140, and memory 142. Implementations of the sensory prosthesis 110 can include more or fewer components than those shown in FIG. 1.

The housing 112 takes any of a variety of different forms, such as a wearable housing (e.g., wearable on a head of the recipient or a wrist of the recipient via a band, strap, magnetic connection, or another fastening technique). In some examples, the sensory prosthesis 110 includes multiple cooperating components disposed in separate housings. An example sensory prosthesis 110 includes an external component (e.g., having components to receive and process sensory data) configured to communicate with an implantable component (e.g., having components to deliver stimulation to cause a sensory percept in the recipient). Where the housing 112 is an implantable housing, the housing 112 is typically constructed from a biocompatible material and is hermetically sealed to resist intrusion of bodily fluid.

The stimulator 120 encompasses one or more components of the sensory prosthesis 110 that provide stimulation to the recipient. The stimulator 120 receives or generates stimulation control signals and generates stimulation based thereon. The stimulator 120 applies the stimulation to the recipient to cause a sensory percept. The stimulation takes any of a variety of forms depending on the type of the sensory prosthesis 110. In many examples, the stimulation includes electrical stimulation (e.g., electrical stimulation of nerve tissue), mechanical stimulation (e.g., bone conducted vibrations), or acoustic stimulation (e.g., air-conducted vibrations directed to the recipient's eardrum). In certain implementations for applicable recipients, the stimulator 120 is configured to stimulate the recipient's nerve cells (e.g., in a manner that bypasses absent or defective cells that normally transduce sensory phenomenon into neural activity to cause a sensory percept in the recipient), in a manner that causes the recipient to perceive one or more components of sensory input data. The illustrated implementation of the stimulator 120 includes a stimulator unit 122 and a stimulator assembly 124.

The stimulator unit 122 is the portion of the stimulator 120 that generates the stimulation. The stimulator unit 122 can also be known as a stimulation generator. Where the stimulator 120 is an electrical stimulator, the stimulator unit 122 generates electrical stimulation signals. Where the stimulator 120 is a mechanical stimulator, the stimulator unit 122 is or includes an actuator configured to generate vibrations. Where the stimulator 120 is an acoustic stimulator, the stimulator unit 122 is or includes a speaker to generate air-conducted vibrations.

The stimulator assembly 124 is the portion of the stimulator 120 via which the stimulation is applied to the recipient. In an example, the stimulator 120 is an electrical stimulator of a cochlear implant and the stimulator assembly 124 is an elongate lead having an array of electrode contacts disposed thereon for delivering the electrical stimulation generated by the stimulator unit 122 to the recipient's cochlea. In another example, the stimulator 120 is a mechanical stimulator and the stimulator assembly 124 is a plate, post, or another component to conduct vibrations from the stimulator unit 122 to a desired portion of the recipient's anatomy. In a still further example, the stimulator 120 is an acoustic stimulator and the stimulator assembly 124 is a structure to channel air conducted vibrations to the recipient's auditory anatomy.

The sensors 130 are one or more components that generate signals based on sensed occurrences, such as data regarding the environment proximate the sensory prosthesis 110, the sensory prosthesis 110 itself, or the recipient. In many examples, the sensors 130 are configured to obtain data for the generation of stimulation via the stimulator 120. In addition or instead, such sensors 130 are used to augment reality, such as using the techniques described herein. In the illustrated example, the sensory prosthesis 110 includes one or more: microphones 132, movement sensors 136, and electrode sensors 138. Additional sensors 130 are also usable.

The one or more microphones 132 include one or more microphones implanted in the recipient or microphones external to the recipient. In an example, the one or more microphones 132 are implemented as transducers that convert acoustic energy (e.g., pressure changes) into electric signals. In certain implementations, the one or more microphones 132 are configured to receive sounds produced internal or external to the recipient. An example implementation of an implantable microphone is configured to resist sensitivity to vibration and acceleration forces. An example of such a microphone is described in U.S. Pat. No. 7,214,179, which was filed Apr. 1, 2005, and which is hereby incorporated by reference in its entirety for any and all purposes. The signals output from the example implantable microphone 132 are filtered or otherwise processed to facilitate improved resulting signals. Examples of such techniques are described in U.S. Pat. No. 8,096,937 (filed Nov. 30, 2007) and U.S. Pat. No. 7,197,152 (filed Feb. 26, 2002), which are both hereby incorporated herein by reference in their entirety for any and all purposes.

In some examples, one or more of the microphones 132 are configured as body noise sensors 134 to sense body noises produced by the recipient. Body noises measurable by the body noise sensors 134 can include, for example, chewing sounds, swallowing sounds, respiration sounds, blood flow sounds, heart beat sounds, or gut sounds, among other sounds. In many examples, the body noise sensor 134 is an implanted, subcutaneous microphone. In other examples, the body noise sensor 134 is an external microphone. The body noise sensors 134 need not exclusively receive body noises. An example body noise sensor 134 is configured to receive sounds produced external to the recipient, which can also happen to receive sounds produced internal to the recipient. In some examples, the data obtained from the body noise sensors 134 is processed to remove the sounds of body noise from stimulation provided by the sensory prosthesis 110. An example of such a technique is described in U.S. Pat. No. 8,472,654, which was filed Oct. 30, 2007, and which is hereby incorporated by reference herein in its entirety for any and all purposes. However, as described elsewhere herein, retaining or enhancing body noises is desirable in certain circumstances to augment the perception of events by the recipient. While many implementations of body noise sensors 134 are configured to detect sound within the range of normal human hearing, certain implementations are configured to detect sound outside of the normal hearing range (e.g., to facilitate detection of body vibrations that may otherwise be inaudible). An example implementation of the body noise sensor 134 includes an accelerometer that detects vibrations (e.g., vibrations outside of a normal hearing range).

Example uses of the one or more microphones 132 (including the body noise sensor 134) include detecting eating noises (e.g., crunching or swallowing sounds), cigarette lighting noises, lighting a cigarette, taking a drag of a cigarette or vape, opening a bottle, opening a can, sipping or drinking from a container, toilet sounds, or vomiting noises, among others. In examples, the one or more microphones 132 (typically implanted microphones 132 or body noise sensors 134) detect noises, such as stomach sounds (e.g. as the stomach anticipates food), tensing of the jaw, chewing, swallowing, breathing, or other noises.

The one or more movement sensors 136 convert motion into electrical signals. Example movement sensors 136 include accelerometers and gyroscopic sensors. In certain implementations, example motion that the movement sensors 136 are configured to detect include: head movement (e.g. indicating alcohol intoxication or vomiting), hand movement (e.g. fork or hand moving toward mouth), or general movement (e.g. walking, riding, driving, or falling).

The one or more electrode sensors 138 are one or more electrodes configured to detect electrical signals. In some examples, the electrode sensors 138 are electrodes of the stimulator assembly 124 that are configured to not only deliver stimulation but also to detect electrical signals. Implementations include one or both of internal and external electrode sensors 138 (e.g., wearable electrodes, such as via a headband).

Example sensors 130 include location sensors. Example location sensors include satellite-based location sensors, such as sensors that are used to determine position based on the GLOBAL POSITIONING SYSTEM (GPS). Further examples of location sensors include components (e.g., one or more antennas) that detect nearby wireless broadcasts, such as WI-FI SSIDs (Service Set Identifiers). Such wireless broadcasts can be useful for determining a current location as well as a current location type. For instance, a restaurant's WI-FI SSID may include certain terms, such as “restaurant”, “bar”, or “café” that are usable as an indication that a recipient is engaging in a behavior associated with a restaurant.

Further example sensors 130 include proximity detectors. An example proximity detector includes a near-field communication sensor, radio-frequency identifier sensor, or hall effect sensor that detects the presence of another device. Example uses of such a proximity sensor include the detection of the recipient's wrist being proximate the recipient's head, which is usable to detect whether the recipient is eating, drinking, smoking or engaging in other behaviors. Other uses include proximity detectors that detect the recipient's proximity to a device, such as a medication dispenser (e.g., a pill bottle or jar).

Additional example sensors 130 include one or more: telecoils, cameras, pupilometers, biosensors (e.g., heart rate or blood pressure sensors), otoacoustic emission sensors (e.g., configured to provide otoacoustic emission signals), EEG (electroencephalography) sensors (e.g., configured to provide EEG signals), glucose sensors (e.g., to provide a blood glucose signal), blood-alcohol sensors (e.g., to provide a blood alcohol signal), or one or more lights sensors (e.g., configured to provide signals relating to light levels).

The illustrated system 100 includes devices discrete from the sensory prosthesis 110 that include the sensors, such as one or more secondary devices 162. In an example implementation, one or more of the secondary devices 162 are communicatively coupled to the sensory prosthesis 110 or the computing device 150, such as via a radiofrequency (e.g., via FM or BLUETOOTH) connection. Example secondary devices 162 include sensors 130 usable to determine behavior of the recipient.

As used herein, sensors 130 further encompass software or hardware components that obtain data. Example software sensors operate on the sensory prosthesis 110 and track data, such as: when the sensory prosthesis 110 is worn by the recipient (e.g., usable to ignore data sensed by a device when the device is not worn by the recipient), when the sensory prosthesis 110 (e.g., an external portion thereof) is removed from the recipient, when one or more sensory prosthesis settings are modified, and a current scene mode in which the sensory prosthesis 110 is operating (e.g., as determined by a scene classifier), among other data. Further software sensors 130 include those that detect or obtain data from software applications (e.g., running on the sensory prosthesis or another device) or connected services, such as a calendar to detect events, smart scales to detect weight, a calorie counter, or a finance tracker to detect purchases. An example finance tracker tracks bank account data usable to determine how much money the recipient spent at a given time and location or on certain kinds of goods (e.g., food or drink).

As mentioned above, in examples the sensors 130 include a scene classifier. A scene classifier is software or hardware that obtains data regarding the environment around the sensory prosthesis (e.g., from one or more of the other sensors 130) and determines a classification of the environment. The classifications can be used to determine settings appropriate for the environment. For example, where the sensory prosthesis 110 is an auditory prosthesis, the scene classifier obtains data regarding the sonic environment around the auditory prosthesis and classifies the sonic environment into one or more possible classifications, such as speech, noise, and music, among other classifications. The sensory prosthesis 110 then uses the classification to automatically alter sensory prosthesis settings 146 to suit the environment. For example, responsive to the scene classifier determining that the sonic environment around the sensory prosthesis 110 is windy, a wind-noise scene can be selected, which modifies settings of the sensory prosthesis 110 to the recipient's perception of wind noise in the provided stimulation. In another example, the scene classifier determines that music is occurring nearby and automatically modifies the sensory prosthesis settings 146 to improve musical reproduction. An example scene classifier is described in US 2017/0359659, filed Jun. 9, 2016, and titled “Advanced Scene Classification for Prosthesis”, which is incorporated by reference herein in its entirety for any and all purposes.

In some examples, the sensors 130 include input components, such as buttons, switches, or user interface elements that obtain data directly from a user of the sensory prosthesis 110. Typically, the user is the recipient of the sensory prosthesis 110, but in some examples, the user is a caregiver of the recipient.

The sensors 130 produce sensor data, which can take any of a variety of different forms depending on the configuration of the sensor 130 that produced the sensor data. Further, the form and character of the sensor data can change as the sensor data is used and moved throughout the system 100. In an example, the sensor data begins as a real-time analog signal that is converted into a real-time digital signal within a sensor 130, which is then transmitted in real-time as packets of data to an application (e.g., of the computing device 150) for batch sending (e.g., non-real-time) to the server 170. Further, the sensor data is processed as the sensor data are used and moved throughout the system 100, such as by being converted into a standardized format and have relevant metadata attached (e.g., timestamps, sensor identifiers, etc.).

The one or more processors 140 are one or more hardware or software processing units (e.g., central processing units) that can obtain and execute instructions, such as to communicate with and control the performance of other components of the sensory prosthesis 110 or the system 100.

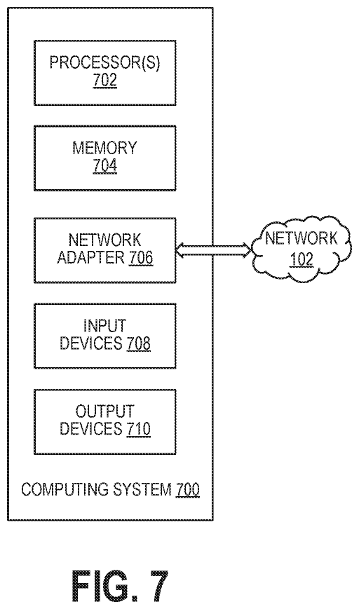

The memory 142 is one or more software- or hardware-based computer-readable storage media operable to store information accessible by the one or more processors 140. Additional details regarding the memory 142 are described in relation to FIG. 7. In the illustrated example, the memory 142 stores instructions 144 and sensory prosthesis settings 146.

The instructions 144 are processor-executable program instructions that, when executed by the one or more processors 140 cause the one or more processors 140 to perform actions or operations, such as those described in the processes herein. The instructions 144 can configure the one or more processors 140 to perform operations.

The sensory prosthesis settings 146 are one or more parameters having values that affect how the sensory prosthesis 110 operates. For example, the settings 146 affect how the sensory prosthesis 110 uses the sensors 130 to receive sensory input from the environment (e.g., using a microphone of the sensory prosthesis 110 to obtain audio input), converts the sensory input into a stimulation signal, and uses the stimulation signal to produce stimulation (e.g., vibratory or electrical stimulation) to cause a sensory percept in the recipient. In an example, the sensory prosthesis settings 146 include a map having minimum and maximum stimulation levels for stimulation channels. The map is used by the sensory prosthesis 110 to control an amount of stimulation to be provided with the stimulator 120. Where the sensory prosthesis 110 is a cochlear implant, the map affects which electrodes of the cochlear implant to stimulate and in what amount based on a received sound input.

In an example, the sensory prosthesis settings 146 include sensory processing settings that modify sensory input before the sensory input is converted into a stimulation signal. Example settings include, in the case of an auditory prosthesis, particular audio equalizer settings can boost or cut the intensity of sound at various frequencies. In examples, the sensory prosthesis settings 146 include a minimum threshold for which received sensory input causes stimulation, a maximum threshold for preventing stimulation above a level which would cause discomfort, gain parameters, intensity parameters (e.g., loudness), and compression parameters. Example sensory prosthesis settings 146 can include settings that affect a dynamic range of stimulation produced by the sensory prosthesis 110. As described above, many of the sensory prosthesis settings 146 affect the physical operation of the sensory prosthesis 110, such as how the sensory prosthesis 110 provides stimulation to the recipient in response to sound input received from the environment. Thus, modifying the sensory prosthesis settings 146 can modify treatment provided by the sensory prosthesis 110. Examples of settings, settings modification, and pre-processing for auditory prostheses are described in U.S. Pat. Nos. 9,473,852 and 9,338,567, which are both incorporated herein by reference for any and all purposes.

### System—Computing Device

The computing device 150 is a computing device associated with the recipient of the sensory prosthesis 110 or a caretaker of the recipient. In many examples, the computing device 150 is a smart phone, smart watch, or heart rate monitor but can take other forms. The illustrated example of the computing device 150 includes a sensory prosthesis application 152.

In the illustrated example, the computing device 150 includes, among other components, one or more sensors 130, memory 142, and a sensory prosthesis application 152. The one or more sensors 130 and memory 142 can be as described above in relation to the sensory prothesis 110.

The sensory prosthesis application 152 is a software application that operates on the computing device 150 and cooperates with the sensory prosthesis 110 directly or via an intermediary device. In an example, the sensory prosthesis application 152 controls the sensory prosthesis 110 (e.g., based on input received from the recipient) and obtains data from the sensory prosthesis 110 and other devices, such as the one or more secondary devices 162. The computing device 150 connects to the sensory prosthesis 110 using, for example, a wireless radiofrequency communication protocol (e.g., BLUETOOTH). The sensory prosthesis application 152 can transmit or receive data from the sensory prosthesis 110 over such a connection. In examples where the sensory prosthesis 110 is an auditory device, the sensory prosthesis application 152 can stream audio to sensory prosthesis 110, such as from a microphone of the computing device 150 or an application running on the computing device 150 (e.g., a video or audio application).

In example implementations herein, one or more of the components of the system 100 cooperate to perform methods augmenting a reality for a recipient, such as by augmenting consumption behavior of the recipient.

### System—Secondary Device

The secondary device 162 is a device separate from the sensory prosthesis 110 that provides sensor data for use in performance of processes and operations described herein. In examples, the secondary device 162 is or includes an additional sensory prosthesis 110 from which data can be obtained. In other examples, the secondary device 162 is a phone, tablet, smart watch, heart rate monitor, wearable EEG, smart ring, or other device having one or more sensors 130. The sensors 130 can be as described above in relation to the sensors 130 of the sensory prosthesis 110. In some examples, the secondary device 162 can obtain data from secondary device sensors 130 and transmit the data to one or more of the other devices or components of the system 100 for processing.

### System—Server

The server 170 is a server computing device remote from the sensory prosthesis 110 and the computing device 150. The server 170 is communicatively coupled to the computing device 150 via the network 102. In many examples, the server 170 is indirectly communicatively coupled to the sensory prosthesis 110 through the computing device 150 (e.g., via the sensory prosthesis application 152). In some examples, the server 170 is directly communicatively coupled to the sensory prosthesis 110 (e.g., via a wireless telecommunications data connection of the sensory prosthesis 110). In certain examples, the sensory prosthesis 110 and the computing device 150 can be considered client devices of the server 170. In some examples, the functionality provided by the server 170 or the components thereof is provided by or located on a device local to the recipient (e.g., the computing device 150 or the sensory prosthesis 110). In some examples, the server 170 communicates with one or more devices via the sensory prosthesis application 152. As illustrated, the server 170 includes a data store 172.

The data store 172 is a component (e.g., hardware memory or one or more data structures stored in hardware memory) for storing data regarding behavior 10. The data store 172 can store data regarding individuals for whom behavior 10 is being monitored. The data regarding the individuals can include various data relevant to techniques described herein. Storable data includes data regarding instances of behavior 10. The data regarding instance of consumption behavior 10 can include representations of the behavior themselves (e.g., sensor data that is the basis of determining that the consumption behavior occurred), data describing the behavior 10 (e.g., frequencies, amount consumed, and other data), and metadata regarding the instances of body noise (e.g., date, time, duration, intensity, normality status) and activity surrounding the instances of the behavior 10. The data can further include notes regarding the instance of the behavior 10 (e.g., authored by a clinician, a caregiver, or the individual).

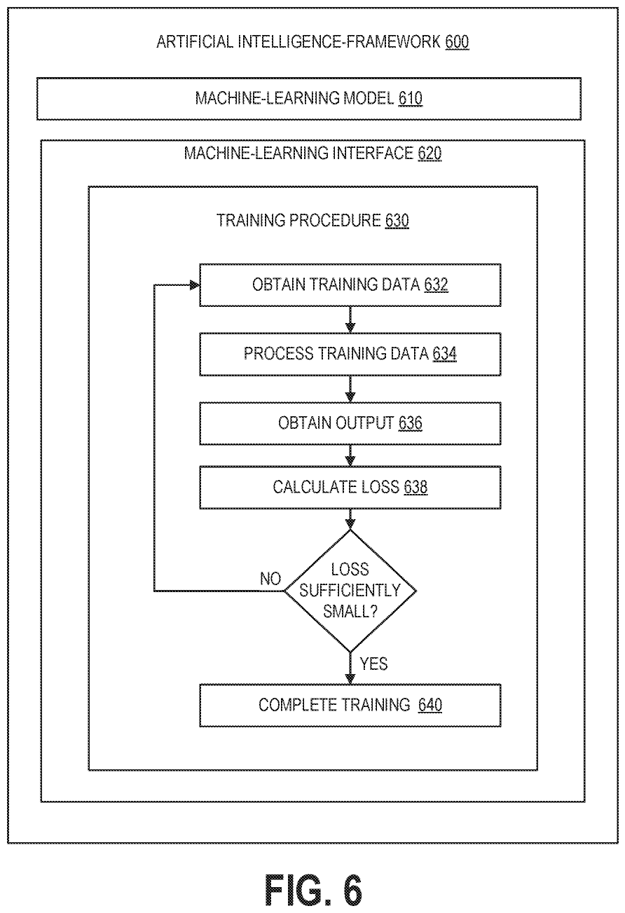

The server 170 can further include one or more processors and memory, which are described in more detail in FIG. 7. The illustrated server 170 further includes an artificial intelligence framework 600, which is described in more detail in relation to FIG. 6. The server 170 can further includes instructions executable to perform one or more of the operations described herein.

### Behavior Indicia

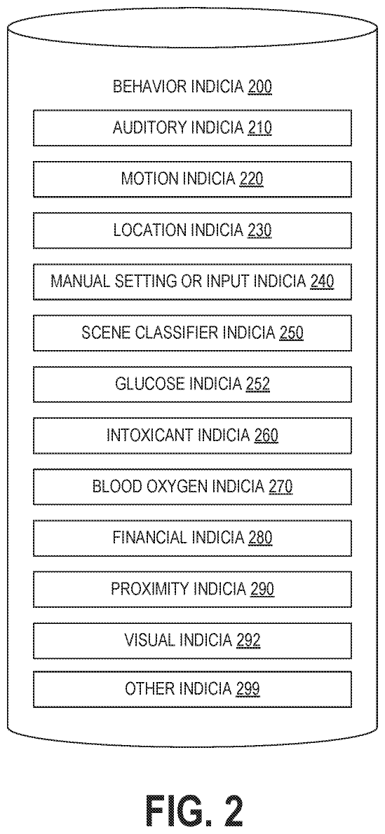

FIG. 2 illustrates example behavior indicia 200. Various examples described herein use behavior indicia 200. As illustrated, example behavior indicia 200 include auditory indicia 210, motion indicia 220, location indicia 230, manual setting or input indicia 240, scene classifier indicia 250, glucose indicia 252, intoxicant indicia 260, blood oxygen sensor indicia 270, financial indicia 280, proximity indicia 290, visual indicia 292, and other indicia 299.

The auditory indicia 210 are indicia of consumption behavior 10 related to audio. Examples of auditory indicia 210 include body noises, such as stomach sounds, jaw tensing sounds, chewing sounds, swallowing sounds, or breathing sounds. Further examples of auditory indicia 210 include ambient sound, such as sounds of a restaurant, bar, or speech. Auditory indicia 210 further include external sound cues, such as the sound of a behavior occurring (e.g., crunching, the sound of lighting a cigarette, the sound of a beer bottle or can being opened, the sound of a toilet, or the sound of vomiting).

The motion indicia 220 are indicia of consumption behavior related to motion. Examples of motion indicia include a movement pattern associated with a consumption behavior, such as the recipient moving his or her hand. Examples of motion indicia further include vibrations associated with chewing. The motion indicia can further indicate the effects of certain consumption behaviors, such as swaying, falling, or other motion (e.g., which may indicate intoxication).

The location indicia 230 are indicia of consumption behavior related to the recipient's location. In an example, location indicia 230 indicates that the recipient is at a particular location associated with particular behaviors, such as a grocery store, coffee shop, smoke shop, dispensary, bar, brewery, or liquor store. Location indicia 230 can further include changes in location over time (e.g., which may be associated with motion 220).

The manual setting or input indicia 240 include indicia relating to, for example, pre-set times, tactile on/off, speech controlled on/off, or a dieting program. The manual setting or input indicia 240 can further include indications from the recipient that he or she is performing a particular consumption behavior.

The scene classifier indicia 250 include indicia of behavior related to a scene classifier. Scene classifier indicia 250 include data regarding a scene in which the sensory prosthesis 110 is or was operating. In an example, the scene classifier indicia indicates that the recipient is using the sensory prosthesis in a particular environment useful in determining whether and what kind of consumption behavior the recipient is engaging. Example scene classifications include speech, noise, speech and noise, music, or quiet, among others.

Glucose indicia 252 includes data related to the recipient's glucose levels (e.g., blood glucose levels). The glucose indicia 252 is usable to indicate that the recipient recently engaged in a behavior (e.g., eating, drinking, or taking insulin) that affected the recipient's glucose level by increasing or decreasing the glucose level.

Intoxication indicia 260 include indicia related to the recipient's level of intoxication, which can broadly encompass the effects of the consumption behavior on the recipient's body. Example intoxication indicia 260 includes indicia of an intoxicant in the recipient's body, such as based on the recipient's blood alcohol level, the presence of nicotine, the presence of caffeine, or the presence of THC (tetrahydrocannabinol), among others. Example intoxicant indicia 260 can indicate that the recipient recently engaged in a behavior (e.g., drinking or smoking) that may intoxicate the recipient.

Blood oxygen indicia 270 include indicia related to the recipient's blood oxygen level. For example, the blood oxygen indicia 270 can indicate that the recipient recently engaged in a behavior that affected the recipient's blood oxygen level.

Financial indicia 280 include indicia related to finance or purchases. An example financial indicia 280 includes bank account data usable to determine how much money the recipient spent at a given time and location or on certain kinds of goods (e.g., food or drink).

Proximity indicia 290 include indicia that the recipient is proximate a particular object or location. For example, the proximity indicia 290 indicates that the recipient recently engaged in a behavior that placed the recipient (or a particular part of the recipient's body) proximate something. Example proximity indicia 290 indicates that the recipient is proximate a medicine dispenser.

Visual indicia 292 include indicia of a behavior based on visual signs. For example, visual indicia 292 can include sights that indicate that the recipient is engaging in a behavior. As a specific example, a visual of a restaurant while the recipient is engaging in a behavior can indicate that the recipient is eating or drinking. As another example, the visual indicia 292 can include visuals of the consumable.

The behavior indicia 200 can include other indicia 299, such as social media posts that describe that the recipient is or has recently engaged in particular behavior. The behavior indicia 200 includes, for example, Internet-of-things indicia (e.g., as obtained from a smart scale), fitness tracker indicia, or services indicia (e.g., calendar events obtained from a calendar service).

### Method

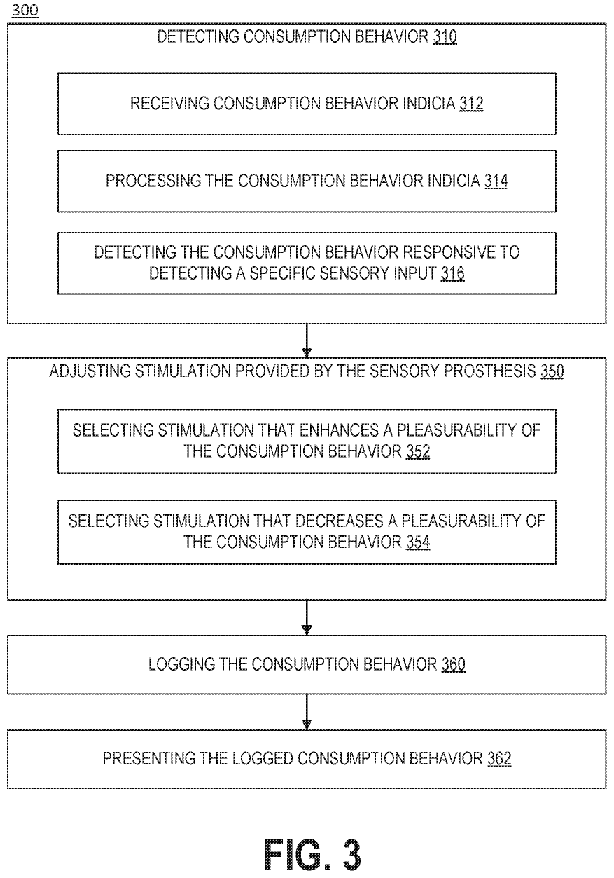

FIG. 3 illustrates an example method 300. In some examples, the method 300 is at least partially performed by a computing device (e.g., computing device 150) such as a phone, a tablet, a wearable computer, a laptop computer, or a desktop computer. In some examples, the method 300 is at least partially performed by the sensory prosthesis 110, such as by one or more processors 140 thereof.

Operation 310 includes detecting a consumption behavior 10 of a recipient of a sensory prosthesis 110. In examples, detecting the consumption behavior 10 includes detecting behavior indicia 200 with one or more of the sensors 130, such as using one or more of the following sensors 130: a microphone, an accelerometer, a location sensor, a manual input, a scene classifier, a glucose sensor, an alcohol sensor, a blood oxygen sensor, a near field communication sensor, or a financial transaction monitor, among others.

Operation 312 includes receiving consumption behavior indicia 200. In an example, the operation 312 includes receiving or obtaining consumption behavior indicia 200 from one or more sensors 130. In an example, the consumption behavior indicia 200 is data usable as a sign to determine whether a consumption behavior occurred. Individual behavior indicia 200 need not be dispositive of whether consumption behavior 10 occurred. In examples, different behavior indicia 200 are combined or analyzed together to determine whether a consumption behavior occurred or whether certain events occurred that make the occurrence of consumption behavior more or less likely.

The consumption behavior indicia 200 is receivable in any of a variety of ways. In at least some examples, the operation 312 includes receiving the consumption behavior indicia 200 from one or more other devices, such as by being pushed the behavior indicia 200 or by pulling the behavior indicia 200 from one or more other devices. In some examples, a first device or sensor 130 obtains or generates the behavior indicia 200, which then transfers processing to another device. In examples, the receiving occurs in real time or is delayed, such as to permit the behavior indicia 200 to be batched.

The operation 312 can include receiving auditory indicia 210. For example, the operation 312 can include receiving auditory indicia 210 with one or more implanted or external microphones 132. In some examples, auditory indicia 210 of a single event is obtained from multiple different microphones 132.

The operation 312 can include receiving motion indicia 220 of the consumption behavior 10 with a motion detector or movement detector 136 (e.g., an accelerometer or gyroscope). An example of receiving the motion indicia 220 includes receiving, from a wrist-worn motion sensor 130, a movement pattern associated with the recipient bringing their hand to their mouth or head. A further example includes receiving motion indicia 220 from a head-worn or implanted motion sensor 130, such as vibrations associated with chewing. A still further example includes receiving motion indicia 220 from a head-worn or implanted motion sensor 130 indicating a swaying of a recipient.

The operation 312 can include receiving location indicia 230 of consumption behavior 10 with a location sensor. In an example, receiving the location indicia 230 includes receiving or detecting the recipient's geographic position with a satellite-based location sensor 130. In another example, the receiving includes determining a kind of location at which the recipient is located, such as by performing a lookup in a database or using a service (e.g., by using an application programming interface of a service provided by a third party) based on a current geographic location of the recipient. Nearby wireless broadcasts can be used to determine the current geographic location, such as by using the names of one or more WIFI SSIDs (Service Set Identifiers). In another example, the location indicia 230 is determined based on the recipient manually or automatically checking-in at a location (e.g., using the computing device 150).

The operation 312 can include receiving manual setting or input indicia 240. In an example, the operation 312 includes receiving times, tactile on/off, speech controlled on/off, or a dieting program. Further examples include receiving manual input from the recipient, such as the recipient indicating that he or she is performing a particular consumption behavior 10. In an example, the recipient uses the sensory prosthesis application 152 to manually specify the consumption behavior 10 in which the recipient is engaging.

The operation 312 can include receiving scene classifier indicia 250 of consumption behavior 10. For example, the scene classifier indicia 250 can include data regarding a scene in which the sensory prosthesis 110 is or was operating. For example, the scene classifier indicia 250 can indicate that the recipient is using the sensory prosthesis 110 in a particular environment that is useful in determining whether and what kind of consumption behavior the recipient is engaging.

The operation 312 can include receiving glucose indicia 252 of consumption behavior 10. For example, the glucose indicia 252 can be obtained from a continuous glucose monitor, a non-invasive glucose monitor, or test strip reader. In a further example, the glucose indicia 252 is obtained from an application (e.g., operating on the computing device 150) that the recipient uses to track his or her glucose levels.

The operation 312 can include receiving intoxicant indicia 260 of consumption behavior 10. In an example, the operation 312 includes receiving intoxicant indicia 260 from a blood alcohol level sensor, a nicotine sensor, a caffeine sensor, or a THC sensor.

The operation 312 can include receiving blood oxygen indicia 270 of consumption behavior 10. For example, the blood oxygen indicia 270 can be obtained from a blood oxygen sensor.

The operation 312 can include receiving proximity indicia 290 of consumption behavior 10. For example, the proximity indicia 290 can be obtained from a proximity sensor, such as a radiofrequency identification receiver, a near field communication receiver, or a Hall effect sensor.

The operation 312 can include receiving financial indicia 280 of consumption behavior 10. The financial indicia 280 can be obtained from, for example, a financial tracking application or service of the recipient and to which the system 100 (or a portion thereof) has been given access.

The operation 312 can include receiving visual indicia 290. For example, the operation 312 can include receiving the visual indicia 290 from one or more cameras or light sensors. Example visual indicia 290 includes one or more videos or still images.

Operation 314 includes processing the consumption behavior indicia 200 to detect the consumption behavior 10. Example implementations of the operation 314 includes performing pre-processing on individual consumption behavior indicia 200. For example, individual consumption behavior indicia 200 is normalized, filtered, smoothed, or otherwise subjected to initial processing to prepare the behavior indicia 200 for further analysis. In examples, the operation 314 includes preforming additional processing to determine whether indicia are indicative of particular consumption behaviors 10. Examples of the operation 314 further include performing a meta-analysis that takes into account indications from multiple different indicia 200 to determine whether a particular consumption behavior 10 occurred.

The operation 314 can include processing received auditory indicia 210 of consumption behavior 10. Examples of processing the auditory indicia 210 include determining whether the auditory indicia 210 includes certain sounds that are associated with consumption behavior 10, such as stomach sounds, jaw tensing sounds, chewing sounds, swallowing sounds, breathing sounds, ambient sounds, (e.g., sounds of a restaurant, bar, or speech), food preparation sounds (e.g., sounds of a package being opened or sounds of food being prepared), sounds of a cigarette being lit, sounds of a beer bottle or can being opened, sounds of a toilet being used, or sounds of vomiting, among other sounds. Identity of the sound is detectable by performing audio analysis on the received auditory indicia 210. For example, the audio analysis can include performing a spectral analysis, a frequency analysis, a volume analysis, or other forms or combinations of audio analysis on the data. The results of the audio analysis are compared with baselines or thresholds to determine whether the audio analysis indicates that the auditory indicia indicate that a particular behavior 10 occurred.

The operation 314 can include processing received motion indicia 220. For example, motion patterns in the motion indicia 220 can be analyzed to determine whether or how well they match motion patterns associated with particular behaviors. A motion indicia 220 being sufficiently close (e.g., within a threshold amount of variation) to a predetermined movement pattern (e.g., a movement pattern associated with eating, smoking, or performing another consumption behavior 10) indicates the occurrence of a particular behavior 10. Further examples of processing include determining or using the presence, absence, or statistical qualities of motion. For example, vibrations detected by a head-worn vibration sensor and having particular qualities (e.g., frequencies or amplitudes being above or below particular thresholds) indicate that a particular behavior is occurring, such as chewing of food in general of even particular kinds of food (e.g., crunchy or soft food).

The operation 314 can include processing received location indicia 230. Example processing includes analyzing the recipient's geographic location (e.g., the coordinates of the recipient) to determine a kind of place at which the recipient is located (e.g., restaurant, park, store, etc.). Activities associated with those kinds of locations or those geographic locations (e.g., activities that the recipient specifically typically engages in or activities that people generally or tend to engage in at those locations) are determined (e.g., using a look-up table or database). The recipient can then be determined to potentially be engaged in behaviors 10 associated with those locations.

The operation 314 can include processing the received manual setting or input indicia 240. For example, the received manual setting or input indicia 240 can be processed to determine whether pre-set times indicate a particular behavior 10, whether tactile on/off settings indicate a particular behavior 10, whether speech controlled settings indicate a particular behavior 10, or if a dieting program indicates a particular behavior 10. Manual input from a recipient can be analyzed to determine whether it indicates that a behavior 10 occurred or is occurring. In an example, the recipient is prompted to answer whether the recipient is engaging in a behavior 10. If the answer is affirmative, then the result of the analysis is that the behavior 10 is occurring, otherwise the behavior 10 is determined to not occur. In other examples, the recipient enters freeform text input, which is processed to determine what (if any) behavior 10 is indicated by the text. The text is analyzed using any of a variety of techniques, such as natural language processing. In a further example, the recipient selects a behavior 10 in which the recipient is engaging from a list of options.

Operation 314 can include processing the received scene classifier indicia 250. For example, different scene classifications are associated with different activities or consumption behaviors. Activities associated with the scene classifications (e.g., activities that the recipient specifically typically engages in or activities that people generally or tend to engage in during those classifications) are then determined (e.g., using a look-up table or database). The recipient is then determined to potentially be engaged in behaviors 10 associated with those scene classifications. For instance, a wind noise scene classification can be associated with behaviors 10 that typically occur outdoors.

Operation 314 can include processing the glucose indicia 252. For example, the recipient's glucose levels or changes in glucose levels that pass certain glucose level thresholds or meet particular glucose level criteria can result in a determination that the recipient recently engaged in a behavior 10 (e.g., eating, drinking, or taking insulin) that affected the recipient's blood glucose level.

Operation 314 can include processing the intoxication indicia 260. For example, the recipient's levels of intoxicants or changes in levels of intoxicants that pass certain intoxicant thresholds or meet particular intoxicant criteria can result in a determination that the recipient recently engaged in a behavior 10 (e.g., smoking or drinking) that intoxicated the recipient.

Operation 314 can include processing the blood oxygen indicia 270. For example, the recipient's blood oxygen levels or changes in blood oxygen levels that pass certain blood alcohol thresholds or meet particular blood alcohol criteria results in a determination that the recipient recently engaged in a behavior that affected the recipient's blood oxygen level.

Operation 314 can include processing the financial indicia 280. For example, different financial indicia 280 are associated with different activities. Activities associated with the financial transactions that the recipient engaged in are determined by analyzing (e.g., using a look-up table or database) transaction records that specify, for example, how much money the recipient spent, where the recipient spent the money, at what time the recipient spent the money, and what the money was spent on (e.g., based on an itemized receipt). Techniques for analyzing such data include natural language processing (e.g., on the name of the business where the recipient spent money) or thresholding (e.g., on the amount), among other techniques. Example analysis includes determining (e.g., using a look-up table or database) what kinds of activities the recipient engaged in or potentially engaged in using the financial indicia 280.

The operation 314 can include processing the proximity indicia 290. For example, the proximity indicia 290 indicates what objects or locations the recipient is near. Then, based on what those objects are, associated behavior 10 is determined. For example, a lookup table is used to determine associated behavior 10 based on the object or restaurant to which the recipient was proximate. For instance, a lookup table includes behaviors such as eating, drinking beverages, and consuming alcohol in association with a restaurant. Further, a number of times that the recipient was proximate the object or duration of the proximity is also able to be analyzed.

The operation 314 can include processing the visual indicia 292. For example, the visual indicia 292 includes an image or frame of a video that is processed to determine one or more objects within the image (e.g., using object detection algorithms, such as neural networks trained on object detection). Then, one or more behaviors 10 associated with the objects can be determined. Further, video or multiple images is usable to determine activities that the recipient is engaged in. For instance, a chip bowl that becomes progressively less full over the course of a video or multiple images can result in a determination that the recipient is engaging in an eating behavior of the contents of the chip bowl.

In some examples, individual indicia are insufficient to be dispositive of a particular behavior. For instance, location indicia 230 that indicates that the recipient is proximate a restaurant might not be sufficient to determine that the recipient is eating or drinking (e.g., the recipient may just be passing by the restaurant when the location is determined). However, that location indicia 230 combined with one or more other indicia may be sufficient to determine that the recipient is eating or drinking (e.g., motion indicia 220 including vibrations indicative of eating). Further, overlap of potential behaviors can be used to indicate that the recipient is engaging in a particular behavior 10.

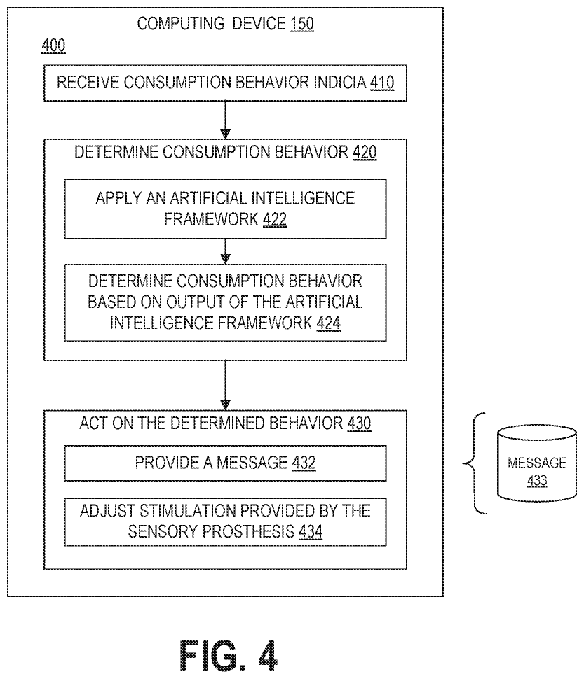

In some examples, operation 314 includes processing the behavior indicia 200 (e.g., individual indicia or combinations thereof) with an artificial intelligence framework, such as is described in more detail below in relation to operations 422 and 424 of FIG. 4. For instance, an artificial intelligence framework includes a decision tree usable to determine the occurrence of one or more behaviors. An example decision tree includes branches based on comparison of one or more of the indicia 200 with thresholds to lead to one or more other branches or leaf nodes (e.g., conclusions regarding whether a particular behavior did or did not occur).

Operation 316 includes detecting the consumption behavior 10 with a specific sensory input. As described in more detail above, certain indicia associated with a person's senses can be associated with particular behavior 10. For instance, a specific sound having particular auditory characteristics is associated with a particular behavior 10 with a high confidence (e.g., a particular fizzing sound detected by a body noise sensor is associated with a recipient drinking a carbonated beverage). As another example, a specific visual having particular characteristics (e.g., as determined based on an object detection algorithm) is specifically associated with food or a particular kind of food (e.g., junk food or healthy food). Responsive to detecting the specific sensory output, a specific consumption behavior 10 is considered detected.

Operation 350 includes adjusting stimulation provided by the sensory prosthesis 110 based on or responsive to the consumption behavior 10, such as to adjust the consumption behavior 10. An example implementation of the operation 350 includes selecting stimulation that enhances a pleasurability of the consumption behavior (operation 352), selecting stimulation that decreases a pleasurability of the consumption behavior (operation 354), or selecting stimulation that otherwise alters the consumption behavior. An example adjustment to the consumption behavior includes adjusting a current behavior that the recipient is engaged in. For example, stimulation can be provided to enhance the auditory experience of eating a chip. Or adjusting the consumption behavior includes adjusting the recipient's long-term habits regarding a particular activity. For instance, stimulation can be provided to decrease the likelihood that the recipient will eat junk food in the future.

In some examples, a lookup table, other data structure, or other arrangement is used to match consumption behaviors 10 with one or more stimulation adjustments. In an example, the detected consumption behavior is used to locate corresponding settings adjustments. Settings adjustments that detract from or enhance the consumption behavior 10 are selectable.

Where the stimulation is auditory stimulation, potential settings adjustments include equalizer settings, volume adjustments, gain adjustments, noise reduction adjustments, settings that enhance or diminish particular portions of an audio signal, other settings, or combinations thereof. Further adjustments having settings that can be modified include adaptive dynamic range optimization, automatic gain control, channel combining, mixing settings, beamforming components, windowing, pre-emphasis control, other adjustments, and combinations thereof.

Where the stimulation is visual stimulation, potential settings adjustments include brightness, contrast, saturation, hue, resolution, dynamic range, other settings adjustments, or combinations thereof.

The operation 350 can include applying various stimulation adjustments. An example stimulation adjustment alters an existing quality of an aspect of the consumption behavior 10 (e.g., the item being consumed). For example, where the consumption behavior is drinking beer and the desired result is less drinking (e.g., discouraging the recipient from drinking), example settings adjustments include reducing high-frequency audio components to decrease perception of fizziness of the beer. As another example, a settings adjustment is selected to mute the sound of a beer bottle or can opening. A further example settings adjustment is to change the hue of the beer in the bottle or glass to appear unnatural or uninviting.

In some examples, the stimulation adjustment adds an aspect to the consumption behavior 10 that is not already present. For instance, stimulation corresponding to an annoying or eerie sensory percept (e.g., auditory percept, visual precept, tactile percept, or vestibular percept) is provided when the recipient engages in the consumption behavior 10 or when the recipient is about to engage in the consumption behavior 10.

Operation 360 includes logging the behavior 10. The behavior 10 is logged in one or more of a variety of locations, such as the sensory prosthesis 110, the computing device 150, the secondary device 162, and the server 170. The behavior 10 is logged for long-term storage and later retrieval. In an example, logging the behavior 10 includes logging data regarding the consumption behavior 10, such as when the consumption behavior 10 occurred and what the consumption behavior 10 is, among other data. In an implementation, the operation 360 includes adding an entry to the data store 172.

Operation 362 includes presenting the logged consumption behavior 10. For example, the component of the system 100 that stores the behavior 10 (e.g., the server 170) receives a request to access logged consumption behavior 10. As a particular example, the recipient can use the computing device 150 to access the consumption behavior 10. The consumption behavior 10 is provided via a user interface that shows, for instance, charts, graphs, and other representations of the consumption behavior of the recipient over a period of time.

### Computing Device Configuration

FIG. 4 illustrates a computing device 150 configured to perform a method 400 that includes various operations. The computing device 150 can include memory having stored thereon instructions that so configure the computing device 150. For instance, the memory can include instructions thereon that, when executed by one or more processors of the computing device 150, cause the one or more processors 140 to perform the one or more operations herein. The computing device 150 can take any of a variety of forms, such as a phone, tablet, wearable computer, laptop computer, desktop computer, or sensory prosthesis 110 (e.g., an external processor thereof).

Operation 410 includes to receive consumption behavior indicia 200. Examples of the operation 410 include one or more aspects described above in relation to operation 312. In an example, the operation 410 includes receiving auditory indicia 210 from a microphone 132. In an example, the operation 410 includes receiving hand movement indicia 220 or head movement indicia 200 from the movement sensor 136. In an example, to receive the consumption behavior indicia 411 regarding the recipient of the sensory prosthesis 110 includes to: receive data from a location sensor, a manual input, a scene classifier, a glucose sensor, an alcohol sensor, a blood oxygen sensor, a near field communication sensor, or a financial transaction monitor.

Operation 420 includes to determine a consumption behavior 10 based on the consumption behavior indicia 200. The operation 420 can include one or more of the aspects described above relating to the detecting of the consumption behavior of operation 310, especially relating to the processing of the consumption behavior indicia in operation 314 and detecting the consumption behavior responsive to detecting a specific sensory input in operation 316. In examples, operation 420 includes operation 422 and operation 424.

Operation 422 includes to apply an artificial intelligence framework 600, and operation 424 includes determining the consumption behavior 10 based on the output of the artificial intelligence framework 600. In an example, the operation 422 includes applying the artificial intelligence framework 600 to the behavior indicia 200. The artificial intelligence framework 600 can include, for example, a machine learning framework trained on consumption behaviors 10. Example implementations of the artificial intelligence framework 600 include one or more algorithms, libraries, pieces of software, or other frameworks that can obtain data, process the data, and provide an output based thereon. Examples implementations of the artificial intelligence framework 600 are configured to receive the received behavior indicia 200 as input and provide, as output, an indication regarding whether the indicia indicates that a particular behavior occurred or is occurring. An example output is a probability that the behavior indicia 200 indicates a consumption behavior 10. The artificial intelligence framework 600 can include one or more human-generated or curated artificial intelligence frameworks 600 configured to receive behavior indicia 200 or other input and provide, as output, an indication of whether the recipient is or has recently performed a particular consumption behavior 10. Artificial intelligence techniques include, for example, decision trees, thresholding, heuristics, scoring, other techniques, or combinations thereof. Additional details regarding the use of artificial intelligence are described in relation to FIG. 6, below.

Operation 430 includes to act on the determined behavior. In an example, the operation 430 includes acting on the determined behavior to adjust the consumption behavior. The operation 430 can include operations 432 and 434.

Operation 432 includes to provide a message 433. The message 433 can include an indication of the consumption behavior. The message can be provided to the recipient, a clinician of the recipient, or a caregiver of the recipient. In an example, the message 433 is a visual or audible message provided by the sensory prosthesis 110, computing device 150, or the secondary device 162 to the recipient. In an example, the message 433 originates from the server 170.

Operation 434 includes to modify a stimulation provided by the sensory prosthesis 110. This operation 434 can include one or more of the aspects described above in relation to operation 350 of FIG. 3.

### Memory

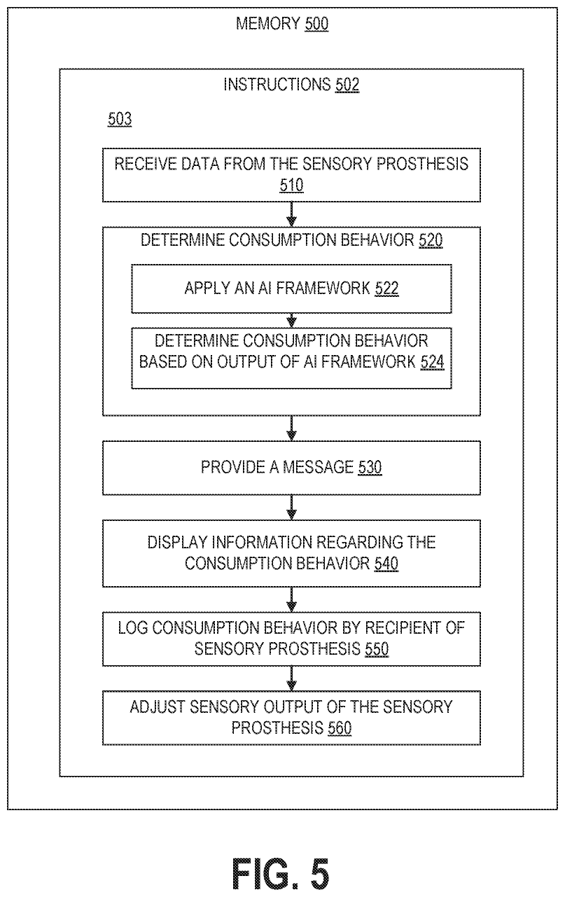

FIG. 5 illustrates example memory 500 having instructions 502 stored thereon that, when executed by one or more processors, cause the one or more processors 140 to perform a method 503 including one or more operations. The memory 500 can be memory of the sensory prosthesis 110 (e.g., an external or implantable component thereof), the computing device 150, the secondary device 162, or the server 170. The memory 500 can be memory of the computing device 150, such as memory of a phone, tablet, wearable computer, laptop computer, or desktop computer.

Operation 510 includes to receive data from the sensory prosthesis 110, such as the indicia 200. The received data can include auditory indicia 210 from one or more implanted microphones, auditory indicia 210 from one or more external microphones, motion indicia 220 (e.g., hand movement indicia or head movement indicia) from one or more accelerometers, location indicia 230, manual setting or input indicia 240, scene classifier indicia 250, glucose indicia 252, intoxication indicia 260, blood oxygen indicia 270, financial indicia 280, proximity indicia 290 (e.g., from a near field communication sensor), visual indicia 292, other indicia, or combinations thereof. In examples, the data is pushed from or pulled from the sensory prosthesis 110. Implementations of the operation 510 can be based on operation 310 of FIG. 3 or operation 410 of FIG. 4.

Operation 520 includes to determine consumption behavior based on the received data. As illustrated, operation 520 includes operations 522 and 524. Operation 522 includes to apply an artificial intelligence framework. Operation 524 includes to determine consumption behavior based on output of the artificial intelligence framework. Example implementations of these operations can include one or more aspects of operations 310, 420, 422, and 422 as described above in relation to FIGS. 3 and 4.

Operation 530 include to provide a message. An example implementation of operation 530 includes one or more aspects described above in relation to operation 432.

Operation 540 include to display information regarding the consumption behavior. In an example, the operation 540 includes displaying information that indicates that a component of the system 100 detected that the recipient is currently or recently engaged in a behavior 10. A further example implementation of operation 540 includes one or more aspects describe above in relation to operation 372.

Operation 550 includes to log consumption behavior by the recipient of the sensory prosthesis 110. In an example, the consumption behavior is logged based on data received by the sensory prosthesis 110. In an example, operation 550 includes one or more aspects of operation 360 of FIG. 3.

Operation 560 includes to adjust sensory output of the sensory prosthesis 110. In an example, the adjustment is to encourage, discourage, or otherwise adjust the consumption behavior. In an example, operation 560 includes one or more aspects of operation 350 of FIG. 3.

### Example Artificial Intelligence Model

FIG. 6 illustrates an example artificial intelligence framework 600 usable with examples herein. For example, one or more of the sensory prosthesis 110, the computing device 150, the server 170, or another device store and operate the artificial intelligence framework 600. The artificial intelligence framework 600 includes software instructions and associated data that implement artificial intelligence capabilities.

In examples, the artificial intelligence framework 600 defines implementations of one or more different artificial intelligence techniques. In an example, the artificial intelligence framework 600 defines a decision tree (e.g., the nodes of the decision tree and the connections therebetween).

In the illustrated example, the artificial intelligence framework 600 includes a machine-learning model 610 and a machine-learning interface 620. One or more aspects of the artificial intelligence framework 600 can be implemented with machine-learning toolkits or libraries, such as: TENSORFLOW by GOOGLE INC. of Mountain View, California; OPENAI GYM by OPENAI of San Francisco, California; or MICROSOFT AZURE MACHINE LEARNING by MICROSOFT CORP. of Redmond, Washington.

The machine-learning model 610 is a structured representation of the learning, such as how learning is achieved and what has been learned. For example, where the machine-learning model 610 includes a neural network, the machine-learning model 610 can define the representation of the neural network (e.g., the nodes of the neural network, the connections between the nodes, the associated weighs, and other data), such as via one or more matrices or other data structures.

The machine-learning interface 620 defines a software interface used in conjunction with the machine-learning model 610. For example, the machine-learning interface 620 can define functions, processes, and interfaces for providing input to, receiving output from, training, and maintaining the machine-learning model 610.

In some examples, the machine-learning interface 620 requires the input data to be preprocessed. In other examples, the machine-learning interface 620 can be configured to perform the preprocessing. The preprocessing can include, for example, placing the input data into a particular format for use by the machine-learning model 610. For instance the machine-learning model 610 can be configured to process input data in a vector format and the data provided for processing can be converted into such a format via the preprocessing. In an example, the interface provides functions that convert the provided data into a useful format and then provide the converted data as input into the machine-learning model 610.

The machine-learning interface 620 can define a training procedure 630 for preparing the machine-learning model 610 for use. The artificial intelligence framework 600 can be trained or otherwise configured to receive data as input and provide an output based thereon. For example, the machine-learning model 610 can be trained to receive data or parameters described herein as input and provide, as output, an indication of whether the provided data is indicative of a potential condition of the recipient's vestibular system or cochlear system. The training procedure 630 can begin with operation 632.

Operation 632 includes obtaining training data. The training data is typically a set of human- or machine-curated data having known training input and desired training output usable to train the machine-learning model 610. In examples herein, the training data can include curated behavior indicia 200 from many different individuals or that is artificially-created and actual or expected output of the machine-learning model 610 for that data (e.g., whether the provided behavior indicia 200 is indicative of a particular behavior 10). For example, the training data can be behavior indicia 200 obtained from individuals known to be engaging in a particular behavior 10. In examples, the data stored in the data store 172 can be used as training data. For example, after a reviewer reviews entries stored in the data store 172, the data can be updated with a reviewer label describing the behavior 10. Such labeled data can be used for training. Following operation 632, the flow can move to operation 634.

Operation 634 includes processing the training data. Processing the training data includes providing the training data as input into the machine-learning model 610. In examples, the training data can be provided as input into the machine-learning model 610 using an associated machine-learning interface 620. Then the machine-learning model 610 processes the input training data to produce an output.

Following operation 634, the flow can move to operation 636. Operation 636 includes obtaining the output from the machine-learning model 610. This can include receiving output from a function that uses the machine-learning model 610 to process input data. Following operation 636, the flow can move to operation 638.

Operation 638 includes calculating a loss value. A loss function is used to calculate the loss value, such as based on a comparison between the actual output of the machine-learning model 610 and the expected output (e.g., the training output that corresponds to the training input provided). Any of a variety of loss functions can be selected and used, such as mean square error or hinge loss. Attributes of the machine-learning model 610 (e.g., weights of connections in the machine-learning model) can be modified based on the loss value, thereby training the model.

If the loss value is not sufficiently small (e.g., does not satisfy a threshold), then the flow can return to operation 632 to further train the machine-learning model 610. This training process continues for an amount of training data until the loss value is sufficiently small or until the training is halted. If the loss value is sufficiently small (e.g., less than or equal to a predetermined threshold), the flow can move to operation 640.

Operation 640 includes completing the training. In some examples, completing the training includes providing the artificial intelligence framework 600 for use in production. For example, the artificial intelligence framework 600 with the trained machine-learning model 610 can be stored on the sensory prosthesis 110, the computing device 150, the server 170, a clinician computing device, or at another location for use. In some examples, prior to providing the artificial intelligence framework 600 for use, the trained machine-learning model 610 is validated using validation input-output data (e.g., data having desired outputs corresponding to particular inputs that are different from the training data), and after successful validation, the artificial intelligence framework 600 is provided for use.

The machine-learning model 610 can include multiple different types of machine-learning techniques. For example, the machine-learning model 610 can define multiple different neural networks, decision trees, and other machine-learning techniques and their connections therebetween. For instance, output of a first neural network can flow to the input of a second neural network with the output therefrom flowing into a decision tree to produce a final output.

### Example Computing System

FIG. 7 illustrates an example of a suitable computing system 700 with which one or more of the disclosed examples can be implemented. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 700 can be a single virtual or physical device operating in a networked environment over communication links to one or more remote devices. The remote device can be a medical device (e.g., the sensory prosthesis 110), a personal computer, a server, a router, a network personal computer, a peer device or other common network node. In examples, the computing device 150, the secondary device 162, the server 170, include one or more components or variations of components of the computing system 700. Further, in some examples, the sensory prosthesis 110 includes one or more components of the computing system 700.

In its most basic configuration, computing system 700 includes one or more processors 702 and memory 704.

The one or more processors 502 include one or more hardware or software processors (e.g., central processing units) that can obtain and execute instructions. The one or more processors 502 can communicate with and control the performance of other components of the computing system 500.

The memory 504 is one or more software- or hardware-based computer-readable storage media operable to store information accessible by the one or more processors 502. The memory 504 can store, among other things, instructions executable by the one or more processors 502 to implement applications or cause performance of operations described herein, as well as other data. The memory 504 can be volatile memory (e.g., RAM), non-volatile memory (e.g., ROM), or combinations thereof. The memory 504 can include transitory memory or non-transitory memory. The memory 504 can also include one or more removable or non-removable storage devices. In examples, the memory 504 can include RAM, ROM, EEPROM (Electronically-Erasable Programmable Read-Only Memory), flash memory, optical disc storage, magnetic storage, solid state storage, or any other memory media usable to store information for later access. In examples, the memory 504 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 504 can include wired media such as a wired network or direct-wired connection, and wireless media such as acoustic, RF, infrared and other wireless media or combinations thereof.

In the illustrated example, the system 500 further includes a network adapter 506, one or more input devices 508, and one or more output devices 510. The system 500 can include other components, such as a system bus, component interfaces, a graphics system, a power source (e.g., a battery), among other components.

The network adapter 506 is a component of the computing system 500 that provides network access. The network adapter 506 can provide wired or wireless network access and can support one or more of a variety of communication technologies and protocols, such as ETHERNET, cellular, BLUETOOTH, near-field communication, and RF (Radiofrequency), among others. The network adapter 506 can include one or more antennas and associated components configured for wireless communication according to one or more wireless communication technologies and protocols.

The one or more input devices 508 are devices over which the computing system 500 receives input from a user. The one or more input devices 508 can include physically-actuatable user-interface elements (e.g., buttons, switches, or dials), touch screens, keyboards, mice, pens, and voice input devices, among others input devices.

The one or more output devices 510 are devices by which the computing system 500 is able to provide output to a user. The output devices 510 can include, displays, speakers, and printers, among other output devices.

### Example Devices

As previously described, the technology disclosed herein can be applied in any of a variety of circumstances and with a variety of different devices. Example devices that can benefit from technology disclosed herein are described in more detail in FIGS. 8-11, below. For example, the techniques described herein can be applied to medical devices (particularly sensory prostheses), such as an implantable stimulation system as described in FIG. 8, a cochlear implant as described in FIG. 9, a bone conduction device as described in FIG. 10, or a retinal prosthesis as described in FIG. 11. The technology can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices, such as smart hearable devices, headphones, augmented reality devices, and virtual reality devices. These different systems and devices can benefit from the technology described herein.

### Example Device—Implantable Stimulator System

FIG. 8 is a functional block diagram of an implantable stimulator system 800 that can benefit from the technologies described herein. In an example, the sensory prosthesis 110 corresponds to the implantable stimulator system 800. The implantable stimulator system 800 includes the wearable device 810 acting as an external processor device and an implantable device 850 acting as an implanted stimulator device. The implantable stimulator system 800 and its components can correspond to the sensory prosthesis 110. In examples, the implantable device 850 is an implantable stimulator device configured to be implanted beneath a recipient's tissue (e.g., skin). In examples, the implantable device 850 includes a biocompatible implantable housing 802. Here, the wearable device 810 is configured to transcutaneously couple with the implantable device 850 via a wireless connection to provide additional functionality to the implantable device 850.

In the illustrated example, the wearable device 810 includes one or more sensors 130, a processor 140, a transceiver 818, and a power source 848. The one or more sensors 130 can be units configured to produce data based on sensed activities. In an example where the stimulation system 800 is an auditory prosthesis system, the one or more sensors 130 include sound input sensors, such as a microphone, an electrical input for an FM hearing system, other components for receiving sound input, or combinations thereof. Where the stimulation system 800 is a visual prosthesis system, the one or more sensors 130 can include one or more cameras or other visual sensors. Where the stimulation system 800 is a cardiac stimulator, the one or more sensors 130 can include cardiac monitors. The processor 140 can be a component (e.g., a central processing unit) configured to control stimulation provided by the implantable device 850. The stimulation can be controlled based on data from the sensor 130, a stimulation schedule, or other data. Where the stimulation system 800 is an auditory prosthesis, the processor 140 can be configured to convert sound signals received from the sensor(s) 130 (e.g., acting as a sound input unit) into signals 851. The transceiver 818 is configured to send the signals 851 in the form of power signals, data signals, combinations thereof (e.g., by interleaving the signals), or other signals. The transceiver 818 can also be configured to receive power or data. Stimulation signals can be generated by the processor 140 and transmitted, using the transceiver 818, to the implantable device 850 for use in providing stimulation.

In the illustrated example, the implantable device 850 includes a transceiver 818, a power source 848, a coil 856, and a stimulator 120 that includes an electronics module 810 and a stimulator assembly 124. The implantable device 850 further includes a hermetically sealed, biocompatible housing enclosing one or more of the components.

The electronics module 810 can include one or more other components to provide sensory prosthesis functionality. In many examples, the electronics module 810 includes one or more components for receiving a signal (e.g., from one or more of the sensors 130) and converting the signal into the stimulation signal 815. The electronics module 810 can further be or include a stimulator unit (e.g., stimulator unit 122). The electronics module 810 can generate or control delivery of the stimulation signals 815 to the stimulator assembly 124. In examples, the electronics module 810 includes one or more processors (e.g., central processing units or microcontrollers) coupled to memory components (e.g., flash memory) storing instructions that when executed cause performance of an operation. In examples, the electronics module 810 generates and monitors parameters associated with generating and delivering the stimulus (e.g., output voltage, output current, or line impedance). In examples, the electronics module 810 generates a telemetry signal (e.g., a data signal) that includes telemetry data. The electronics module 810 can send the telemetry signal to the wearable device 810 or store the telemetry signal in memory for later use or retrieval.

The stimulator assembly 124 can be a component configured to provide stimulation to target tissue. In the illustrated example, the stimulator assembly 124 is an electrode assembly that includes an array of electrode contacts disposed on a lead. The lead can be disposed proximate tissue to be stimulated. Where the system 800 is a cochlear implant system, the stimulator assembly 124 is insertable into the recipient's cochlea. The stimulator assembly 124 can be configured to deliver stimulation signals 815 (e.g., electrical stimulation signals) generated by the electronics module 810 to the cochlea to cause the recipient to experience a hearing percept. In other examples, the stimulator assembly 124 is a vibratory actuator disposed inside or outside of a housing of the implantable device 850 and configured to generate vibrations. The vibratory actuator receives the stimulation signals 815 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 818 can be components configured to transcutaneously receive and/or transmit a signal 851 (e.g., a power signal and/or a data signal). The transceiver 818 can be a collection of one or more components that form part of a transcutaneous energy or data transfer system to transfer the signal 851 between the wearable device 810 and the implantable device 850. Various types of signal transfer, such as electromagnetic, capacitive, and inductive transfer, can be used to usably receive or transmit the signal 851. The transceiver 818 can include or be electrically connected to the coil 856.

The coils 856 can be components configured to receive or transmit a signal 851, typically via an inductive arrangement formed by multiple turns of wire. In examples, in addition to or instead of a coil, other arrangements are used, such as an antenna or capacitive plates. The magnets can be used to align respective coils 856 of the wearable device 810 and the implantable device 850. For example, the coil 856 of the implantable device 850 is disposed in relation to (e.g., in a coaxial relationship) with an implantable magnet set to facilitate orienting the coil 856 in relation to the coil 856 of the wearable device 810 via the force of a magnetic connection. The coil 856 of the wearable device 810 can be disposed in relation to (e.g., in a coaxial relationship) with a magnet set.

The power source 848 can be one or more components configured to provide operational power to other components. The power source 848 can be or include one or more rechargeable batteries. Power for the batteries can be received from a source and stored in the battery. The power can then be distributed to the other components of the implantable device 850 as needed for operation.

As should be appreciated, while particular components are described in conjunction with FIG. 8, technology disclosed herein can be applied in any of a variety of circumstances. The above discussion is not meant to suggest that the disclosed techniques are only suitable for implementation within systems akin to that illustrated in and described with respect to FIG. 8. In general, additional configurations can be used to practice the methods and systems herein and/or some aspects described can be excluded without departing from the methods and systems disclosed herein.

### Example Device—Cochlear Implant

FIG. 9 illustrates an example cochlear implant system 910 that can benefit from use of the technologies disclosed herein. For example, the cochlear implant system 910 can be used to implement the sensory prosthesis 110. The cochlear implant system 910 includes an implantable component 944 typically having an internal receiver/transceiver unit 932, a stimulator unit 920, and an elongate lead 918. The internal receiver/transceiver unit 932 permits the cochlear implant system 910 to receive signals from and/or transmit signals to an external device 950. The external device 950 can be a button sound processor worn on the head that includes a receiver/transceiver coil 930 and sound processing components. Alternatively, the external device 950 can be just a transmitter/transceiver coil in communication with a behind-the-ear device that includes the sound processing components and microphone.

The implantable component 944 includes an internal coil 936, and preferably, an implanted magnet fixed relative to the internal coil 936. The magnet can be embedded in a pliable silicone or other biocompatible encapsulant, along with the internal coil 936. Signals sent generally correspond to external sound 913. The internal receiver/transceiver unit 932 and the stimulator unit 920 are hermetically sealed within a biocompatible housing, sometimes collectively referred to as a stimulator/receiver unit. Included magnets can facilitate the operational alignment of an external coil 930 and the internal coil 936 (e.g., via a magnetic connection), enabling the internal coil 936 to receive power and stimulation data from the external coil 930. The external coil 930 is contained within an external portion. The elongate lead 918 has a proximal end connected to the stimulator unit 920, and a distal end 946 implanted in a cochlea 940 of the recipient. The elongate lead 918 extends from stimulator unit 920 to the cochlea 940 through a mastoid bone 919 of the recipient. The elongate lead 918 is used to provide electrical stimulation to the cochlea 940 based on the stimulation data. The stimulation data can be created based on the external sound 913 using the sound processing components and based on sensory prosthesis settings.

In certain examples, the external coil 930 transmits electrical signals (e.g., power and stimulation data) to the internal coil 936 via a radio frequency (RF) link. The internal coil 936 is typically a wire antenna coil having multiple turns of electrically insulated single-strand or multi-strand platinum or gold wire. The electrical insulation of the internal coil 936 can be provided by a flexible silicone molding. Various types of energy transfer, such as infrared (IR), electromagnetic, capacitive and inductive transfer, can be used to transfer the power and/or data from external device to cochlear implant. While the above description has described internal and external coils being formed from insulated wire, in many cases, the internal and/or external coils can be implemented via electrically conductive traces.

### Example Device—Bone Conduction Device

FIG. 10 is a view of an example of a bone conduction device 1000 that can benefit from use of the technologies disclosed herein. For example, the bone conduction device 1000 corresponds to the sensory prosthesis 110. The bone conduction device 1000 is positioned behind an outer ear 1001 of a recipient of the device. The bone conduction device 1000 includes a sound input element 1026 to receive sound signals 1007. The sound input element 1026 can be a microphone, telecoil or similar. In the present example, the sound input element 1026 is located, for example, on or in the bone conduction device 1000, or on a cable extending from the bone conduction device 1000. Also, the bone conduction device 1000 comprises a sound processor (not shown), a vibrating electromagnetic actuator and/or various other operational components.

More particularly, the sound input element 1026 converts received sound signals into electrical signals. These electrical signals are processed by the sound processor. The sound processor generates control signals that cause the actuator to vibrate. In other words, the actuator converts the electrical signals into mechanical force to impart vibrations to a skull bone 1036 of the recipient. The conversion of the electrical signals into mechanical force can be controlled by input received from a user.

The bone conduction device 1000 further includes a coupling apparatus 1040 to attach the bone conduction device 1000 to the recipient. In the illustrated example, the coupling apparatus 1040 is attached to an anchor system (not shown). An exemplary anchor system (also referred to as a fixation system) includes a percutaneous abutment fixed to the skull bone 1036. The abutment extends from the skull bone 1036 through muscle 1034, fat 1028 and skin 1032 so that the coupling apparatus 1040 can be attached thereto. Such a percutaneous abutment provides an attachment location for the coupling apparatus 1040 that facilitates efficient transmission of mechanical force. Alternative coupling arrangements can be used including non-percutaneous coupling using, for example, a headband.

### Example Device—Retinal Prosthesis

FIG. 11 illustrates a retinal prosthesis system 1101 that comprises an external device 1110, a retinal prosthesis 1100 and a mobile computing device 1103. The retinal prosthesis system 1101 can correspond to the sensory prosthesis 110. The retinal prosthesis 1100 comprises a processing module 1125 and a retinal prosthesis sensor-stimulator 1190 is positioned proximate the retina 1191 of a recipient. The external device 1110 and the processing module 1125 can both include transmission coils 1156 aligned via respective magnet sets. Signals 1151 can be transmitted using the coils 1156.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190, and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

Similar to the above examples, the retinal prosthesis system 1101 may be used in spatial regions that have at least one controllable network connected device associated therewith (e.g., located therein). As such, the processing module 1125 includes a performance monitoring engine 1127 that is configured to obtain data relating to a “sensory outcome” or “sensory performance” of the recipient of the retinal prosthesis 1100 in the spatial region. As used herein, a “sensory outcome” or “sensory performance” of the recipient of a sensory prosthesis, such as retinal prosthesis 1100, is an estimate or measure of how effectively stimulation signals delivered to the recipient represent sensor input captured from the ambient environment.

Data representing the performance of the retinal prosthesis 1100 in the spatial region is provided to the mobile computing device 1103 and analyzed by a network connected device assessment engine 1162 in view of the operational capabilities of the at least one controllable network connected device associated with the spatial region. For example, the network connected device assessment engine 1162 may determine one or more effects of the controllable network connected device on the sensory outcome of the recipient within the spatial region. The network connected device assessment engine 1162 is configured to determine one or more operational changes to the at least one controllable network connected device that are estimated to improve the sensory outcome of the recipient within the spatial region and, accordingly, initiate the one or more operational changes to the at least one controllable network connected device.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A method comprising:
detecting a consumption behavior of a recipient of a sensory prosthesis; and
adjusting stimulation provided by the sensory prosthesis to adjust the consumption behavior.

2. The method of claim 1, wherein detecting the consumption behavior includes:
receiving consumption behavior indicia from one or more sensors; and
processing the consumption behavior indicia to detect the consumption behavior.

3. The method of claim 2, wherein obtaining consumption behavior indicia from one or more sensors includes:
receiving auditory indicia of the consumption behavior with an implanted microphone.

4. The method of claim 2, wherein obtaining consumption behavior indicia from one or more sensors includes:
receiving motion indicia of the consumption behavior with a motion detector.

5. The method of claim 1, wherein adjusting the stimulation provided by the sensory prosthesis to adjust the consumption behavior includes:
selecting stimulation that enhances a pleasurability of the consumption behavior;
selecting stimulation that decreases a pleasurability of the consumption behavior; or
selecting stimulation that otherwise alters the consumption behavior.

6. The method of claim 1, further comprising:
logging the consumption behavior; and
presenting the logged consumption behavior.

7. The method of claim 1, wherein detecting the consumption behavior includes detecting the consumption behavior responsive to detecting a specific sensory input.

8. The method of claim 1,
wherein the consumption behavior is at least one of an eating behavior, a drinking behavior, a vaping behavior, or a smoking behavior.

9. A system comprising:
a sensory prosthesis of a recipient;
a microphone;
a movement sensor; and
a computing device configured to:
receive, from the microphone and the movement sensor, consumption behavior indicia regarding the recipient;
determine a consumption behavior of the recipient based on the consumption behavior indicia; and
act on the determined behavior to adjust the consumption behavior.

10. The system of claim 9, wherein to determine the consumption behavior based on the consumption behavior indicia includes to:
apply an artificial intelligence framework to the consumption behavior indicia; and
determine the behavior based on an output of the artificial intelligence framework.

11. The system of claim 10, wherein the artificial intelligence framework is a machine learning framework trained on consumption behaviors.

12. The system of claim 9, wherein to act on the determined consumption behavior includes to:
provide a message to the recipient, the message including an indication of the consumption behavior; or
adjust a stimulation provided by the sensory prosthesis.

13. The system of claim 9, wherein to receive the consumption behavior indicia regarding the recipient of the sensory prosthesis includes to:
receive auditory indicia from the microphone; and
receive hand movement indicia or head movement indicia from the movement sensor.

14. The system of claim 9, wherein the microphone is an implanted microphone.

15. The system of claim 9,
wherein to receive the consumption behavior indicia regarding the recipient of the sensory prosthesis includes to: receive data from a location sensor, a manual input, a scene classifier, a glucose sensor, an alcohol sensor, a blood oxygen sensor, a near field communication sensor, or a financial transaction monitor.

16. A computer-readable medium having instructions stored thereon that, when executed by one or more processors cause the one or more processors to:
log a consumption behavior by a recipient of a sensory prosthesis based on data obtained by the sensory prosthesis; and
display information regarding the consumption behavior.

17. The computer-readable medium of claim 16, wherein the instructions further cause the one or more processors to:
receive the data from the sensory prosthesis; and
determine the consumption behavior based on the received data.

18. The computer-readable medium of claim 17, wherein to determine the consumption behavior based on the received data includes to:
apply an artificial intelligence framework to the data from the sensory prosthesis; and
determine the consumption behavior based on an output of the artificial intelligence framework.

19. The computer-readable medium of claim 16, wherein the instructions further cause the one or more processors to:
adjust sensory output of the sensory prosthesis responsive to a sensory input to at least one of encourage, discourage, or adjust the consumption behavior of the recipient of the sensory prosthesis.

20. The computer-readable medium of claim 16, wherein the instructions further cause the one or more processors to provide a message to the recipient, the message including an indication of the consumption behavior.
